(19) United States
(12) Patent Application Publication
(10) Pub. No.: US 2020/0237731 A1
(43) Pub. Date: Jul. 30, 2020

(54) USE OF CLOXACILLIN TO INHIBIT/PREVENT BIOFILM FORMATION

(71) Applicant: BIOFILM CONTROL, SAINT BEAUZIRE (FR)

(72) Inventors: Christian PROVOT, LE CENDRE (FR); Thierry BERNARDI, PERIGNAT LES SARLIEVE (FR)

(21) Appl. No.: 16/755,265

(22) PCT Filed: Jul. 23, 2018
(86) PCT No.: PCT/FR2018/051880

§ 371 Date: Apr. 10, 2020

(30) Foreign Application Priority Data
Oct. 18, 2017 (FR) 1759783

(51) Int. Cl. A61K 31/431; A61P 31/04

(52) U.S. Cl. CPC A61K 31/431; A61P 31/04

(57) ABSTRACT

A pharmaceutical composition comprising cloxacillin for use as a medicament for the prevention of bacterial biofilm formation. Use of cloxacillin for the prevention and/or inhibition of bacterial biofilm formation on a surface.

### TECHNICAL FIELD

The present disclosure relates to a pharmaceutical composition comprising cloxacillin for use as a drug for the prevention of bacterial biofilm formation.

The present disclosure also relates to the use of cloxacillin for the prevention and/or inhibition of bacterial biofilm formation on a surface.

The present disclosure also relates to a medical device comprising cloxacillin on its surface.

The present disclosure finds an application in particular in the pharmaceutical and medical fields.

### BACKGROUND

Biofilms are made up of different layers of bacteria or microorganisms, often contained in a solid matrix. They grow to form microbial communities, one of whose properties is to adhere to submerged surfaces. This adhesion is either non-specific (adherence) or specific (actual adhesion) (Costerton et al., “Bacterial Biofilms: a common cause of persistent infections”. Science 1999; 284, 1318-1322):
Reversible adherence or adhesion: The present microorganisms approach the surfaces by gravimetry, Brownian movements or by chemotaxis if they have flagella. During this first stage of attachment, involving only purely physical phenomena and weak physicochemical interactions, the microorganisms can still be easily detached.
Adhesion: This slower stage involves higher energy interactions as well as the microbial metabolism and the cellular appendages of the microorganism (flagella, pilis, etc.). Adhesion is an active and specific phenomenon. Some first colonizers will attach themselves irreversibly to the surface, thanks in particular to the synthesis of exopolysaccharides (EPS). This process is relatively slow and depends on environmental factors and the microorganisms present.

These biofilms are ubiquitous in many areas, where they pose health risks and cause relatively significant damage.

In human health for example, biofilms are responsible for infections that are increasingly difficult to control: throughout the head and neck area (ear canal, nasal mucous membrane, conjunctiva of the eye, etc.), on the teeth (appearance of tartar, cavities, etc.), on the bronchial tubes, the lungs (in patients with pneumopathies, cystic fibrosis, etc.), in the urogenital tract, and in wounds. They are also the cause of most nosocomial pathologies (more than 10 000 deaths per year) by forming at the level of catheters or implants (heart valves, artificial hips, urinary catheters, etc.) (Costerton et al., “Bacterial Biofilms: a common cause of persistent infections”. Science 1999; 284, 1318-1322).

Bacterial biofilms that develop on implants or during chronic infections are reservoirs of pathogens that are at the origin of many nosocomial infections.

Despite the implementation of preventive measures, biofilms are difficult to prevent and eradicate because of their characteristic tolerance to prescribed therapeutic doses of antibiotics, and even high doses of antibiotics. This tolerance allows microorganisms to persist (hence the name persistence) and then develop resistance within the biofilm (appearance of mutations, exchange of resistance genes) (Hoiby, N. et al.; (2010) Antibiotic resistance of bacterial biofilms. International Journal of Antimicrobial Agents, 35, 322-332).

Osteoarticular infections are infections that affect bones and/or joints that are frequently caused by staphylococci. There are also complex osteoarticular infections that occur particularly in individuals with joint prostheses or implantable medical devices. These infections are particularly difficult to treat as they frequently show tolerance (also called recalcitrance) to antibiotics, especially due to the presence of biofilm, as well as resistance as described above.

In particular, it is known that the surface of prostheses, especially joints, is a favorable territory for the proliferation of bacteria and the formation of biofilm.

It has been shown, based on a cohort of 124 patients with implant-related osteomyelitis due to Staphylococcus epidermidis infection (responsible for approximately 20% of osteoarticular infections) that cure rates decrease as the biofilm-forming capacity of the isolates increases. In particular, the cure rate for osteoarticular infections is 84% for infections caused by a non-biofilm-forming strain/bacteria, the cure rate is 76% for a weak biofilm-forming strain/bacteria, and the cure rate is 60% for the most pronounced biofilm-forming strain/bacteria (Morgenstern et al., J Orthop Res. 2016 Noveber; 34(11):1905-1913). This analysis of the failure rate should be linked to an in vitro study showing the ineffectiveness of gentamicin treatment of bone cement on biofilm formation by clinical isolates of S. aureus (Tunney et al., J Orthop Res. 2007 January; 25(1):2-10). Also, a study compiling in vitro, in vivo analyses and strain DNA sequencing suggests that chronicity of S. aureus in osteoarticular infections is particularly associated with biofilm formation (Trouillet-Assant et al., Cell Microbiol. 2016 October; 18(10):1405-14). Finally, a study of 66 patients led to the conclusion that the treatment failure rate for osteoarticular infections is 24.2% (Valour et al. BMC Infect Dis. 2014 Aug. 16; 14:44), despite the use of antibiotics supposedly adapted to the pathogen.

A study of ampicillin and penicillin (of the beta-lactam family) on a possible preventive action against biofilm formation in polystyrene microplate has been carried out (Singh and Mishra “Synergistic and antagonistic actions of antibiotics against biofilm forming Staphylococcus aureus”, Asian Journal of Plant Science and Research, 2012). However, no particular application and/or use for these two compounds was considered, the authors concluding that the compounds tested individually are not sufficiently active, whereas only combinations of beta-lactam antibiotics with macrolides could be effective in vitro. This paper does not describe any in vivo applications or present any evidence of any in vivo effect.

In addition, it has been shown that an antibiotic, at a dose below its MIC (sub-MIC), can promote bacterial attachment. For example, aminoglycosides at sub-MIC doses induce adhesion of strains of Pseudomonas aeruginosa and Escherichia coli (Hoffman et al., 2005, Nature, 436: 1171-1175), metronidazole increases biofilm formation by Clostridium difficile (Vuotto et al., 2016, Pathod Dis, 74, doi: 10.1093/femspd/ftv114), amoxicillin at sub-MIC doses induces attachment of S. aureus strain USA 300 (Mlynek et al., 2016, Antimicrob Agents Chemother., 60:2639-51), but more importantly, oxacillin, like vancomycin, at sub-MIC doses induces biofilm formation by S. aureus (Mirani et al., 2011, J Basic Microbiol., 51:191-5). This effect is widely observed for many bacterial strains, by many antibiotics (reviewed by Kaplan, 2011, Int J Artif Organs 34: 737-751). It is known that the serum level or concentration of a substance between two administrations varies over time around its effective concentration, i.e. first above, then within the effective concentration range, and finally, prior to re-administration, below the effective concentration range. Thus, for an antibiotic, there is a time during which the concentration of the antibiotic is lower than the minimum inhibitory concentration (MIC), thus favoring the attachment of bacteria and possibly the formation of biofilm.

There is therefore a real need to find a novel means/compound to overcome these defects, disadvantages and obstacles of the prior art, in particular a means/compound to prevent biofilm formation and/or degrade biofilms.

In particular, there is therefore a real need to find a novel means/compound to overcome these defects, disadvantages and obstacles of the prior art, in particular a means/compound to prevent the formation of biofilm and/or to degrade the biofilms present in particular on the surface of prostheses and/or implantable medical devices.

There is therefore a real need to find a novel means/compound to overcome these defects, disadvantages and obstacles of the prior art, in particular a means/compound to prevent biofilm formation and/or degrade biofilms.

In particular, there is a real need to find a novel means/composite to overcome these defects, disadvantages and obstacles of the prior art, in particular a means/composite to prevent the formation of biofilm and/or to degrade the biofilms present in particular on the surface of prostheses and/or implantable medical devices.

There is also a real need to find a novel way/compound to prevent/treat biofilms of osteoarticular infections, wounds including superficial, deep wounds, diabetic foot infections, burns, bedsores.

In particular, there is therefore a real need to find a novel means/compound to overcome these defects, disadvantages and obstacles of the prior art, in particular a means/compound to prevent the formation of biofilm in wounds and/or infections, for example osteoarticular.

### BRIEF SUMMARY

The present disclosure relates to a pharmaceutical composition comprising cloxacillin for use as a medicament for the prevention of bacterial biofilm formation.

The present disclosure also relates to the use of cloxacillin for the prevention and/or inhibition of bacterial biofilm formation on a surface.

### BRIEF DESCRIPTION OF THE FIGURES

The appended figures are provided by way of illustration.

FIG. 1 includes a digitized image (scan) of a 96-well plate and a diagram showing the change in biofilm index (BFI) (y-axis) as a function of cloxacillin concentration in μg/mL. FIG. 1A is a digitized image of the plate after magnetization and represents the raw result of the Antibiofilmogram of strain S14 and strain S39, lines E and A correspond respectively to strains S14 and S39 grown in the presence of different cloxacillin concentrations. Lines B, C and D correspond to other strains not mentioned here. Line F corresponds to controls in the absence of strain (normal presence of a spot), wells H1 and H2 correspond to strain S14 alone, and wells H9 and H10 correspond to strain S39 alone. Each column from 1 to 12 corresponds to different concentrations in μg/mL of cloxacillin i.e. from column 1 to column 12): 32, 16, 8, 7, 6, 5, 4, 3, 2, 1, 0.5, 0.25 μg/mL.

FIG. 2 shows confocal microscopic images of the surface of the bottom of the well in which bacteria have been cultured, the light spots on these photographs correspond to the bacteria forming the biofilm. The diagram represents the percentage of surface area covered by biofilm (y-axis). FIG. 2A shows the results for bacterial strain S14 cultured without antibiotics (Control) with a cloxacillin concentration below the MIC (<MIC), a cloxacillin concentration equal to its MIC (MIC), a concentration between the MIC and the biofilm inhibitory concentration (<cmib), and a concentration equal to the biofilm inhibitory concentration (cmib).

FIG. 2B corresponds to the results obtained with the bacterial strain S39 grown without antibiotic (Control) with a cloxacillin concentration below the MIC (<MIC), a cloxacillin concentration equal to its MIC (MIC), a concentration between the MIC and the biofilm inhibitory concentration (<cmib) and a concentration equal to the biofilm inhibitory concentration (cmib).

FIG. 3 shows diagrams corresponding to the enumeration of bacteria attached to an implanted catheter in mice as a function of the presence or absence of cloxacillin. The y-axis is the log decimal number of colony forming units (Logio CFU) per gram catheter (Logio CFU/gram catheter). The x-axis is the time after infection with the strain and as a function of the cloxacillin treatment: without cloxacillin (Control) after 12 hours (H12), 24 hours (H24), after 30 hours (H30) or with cloxacillin (Cloxa) treatment at different concentrations (< or >). FIG. 3A shows the diagram obtained with strain S14, with cloxacillin administered at a concentration of 3 mg/kg (<2) or 15 mg/kg (>2). FIG. 3B represents the diagram obtained with strain S39, with cloxacillin administered at a concentration of 4 mg/kg (<4) or 25 mg/kg (>4).

FIG. 4A shows the measurement of the healing score in infected mice treated with either cloxacillin corresponding to the MIC of strain S14 (3 mg/kg), cloxacillin corresponding to the MICb dose (15 mg/kg), or saline (vehicle).

FIG. 4B shows the measurement of the healing score in uninfected (control) mice treated with either cloxacillin corresponding to the MIC of strain S14 (Infected/3 mg/kg), cloxacillin corresponding to the MICb dose (Infected/15 mg/kg), or saline (Infected/Control).

FIG. 4C shows the count of bacteria in the wound. Their number is conventionally expressed in colony-forming units (CFU) corresponding to the number of colonies counted on a Petri dish.

### DETAILED DESCRIPTION

The present disclosure resolves the disadvantages and obstacles of the prior art through the use of cloxacillin at a concentration higher than 4 mg/L, for example from 4 mg/L to 16 mg/L.

The present disclosure also resolves the disadvantages and obstacles of the prior art through the use of cloxacillin at a concentration higher than 25 mg/kg, for example from 25 to 100 mg/kg, preferably from 25 to 50 mg/kg.

The inventors have demonstrated in a surprising and unexpected way that use at a concentration higher than 4 mg/L, for example from 4 mg/L to 16 mg/L, allow advantageously to inhibit the formation and/or development of biofilms on a surface.

The present disclosure also resolves the disadvantages and obstacles of the prior art by using cloxacillin at a dose higher than 25 mg/kg, for example from 25 to 100 mg/kg, preferably from 25 to 50 mg/kg, as a medicament for inhibiting the formation and/or development of biofilms on a surface.

The inventors demonstrated in a surprising and unexpected way that the use of cloxacillin as a medicament at a dose higher than 25 mg/kg, for example 25 to 100 mg/kg, preferentially 25 to 50 mg/kg, is advantageous in inhibiting the formation and/or development of biofilms.

In this document, mg/kg refers to a dose in milligrams per kilogram of the individual to be treated.

The present disclosure also resolves the disadvantages and obstacles of the prior art by providing a pharmaceutical composition comprising cloxacillin at a dose higher than 4 mg/L, for example from 4 mg/L to 16 mg/L, for use as a medicament for the prevention and/or inhibition of bacterial biofilm formation.

The inventors are the first to have surprisingly demonstrated an inhibition of biofilm formation by cloxacillin. In particular, the inventors are the first to have surprisingly demonstrated an inhibition of biofilm formation by cloxacillin at a concentration or in an amount used which is totally different and independent of the known concentration/amount and/or useful for use as a bactericide or “bacteria killer”.

In particular, the inventors have demonstrated, inter alia, an inhibition of biofilm formation under the in vitro conditions used, comprising a short incubation (4 h) of clinical strains of Staphylococcus aureus, in the presence of cloxacillin from the beginning of incubation. As demonstrated in the examples, the inventors demonstrated and characterized the effect of cloxacillin on the initial formation of biofilm, adherence and adhesion steps. In the same way that antibiotic susceptibility tests can define, over a range of doses tested, a dose corresponding to the minimum inhibitory concentration (MIC), the inventors have demonstrated that above a particular concentration and/or in a range of concentrations, cloxacillin inhibits biofilm formation. The minimum concentration to inhibit biofilm formation is also referred to in the text as the biofilm minimum inhibitory concentration (MICb). The inventors have also demonstrated that the MICb can vary, as the MIC, depending on the strain, however, they have also demonstrated that the variation can be within a range of values that advantageously allows the use of cloxacillin to inhibit biofilm formation, without the need for characterization, for example MIC and/or MICb determination, of the strain, for example by using a concentration and/or dose corresponding to a frequently found MICb.

Cloxacillin is an antibiotic of the β-lactam family, of the group of penicillins of group M. Its action is bactericidal, i.e. it will kill bacteria, and it is used for that purpose. It is used to treat infections caused by penicillinase-producing staphylococci, including pneumococci, group A hemolytic streptococci β-hemolytics, and penicillin G-sensitive or resistant staphylococci. Its usefulness is therefore to bypass a penicillin resistance mechanism for the sole purpose of killing germs.

Cloxacillin is a β-lactamine of the following formula (I):

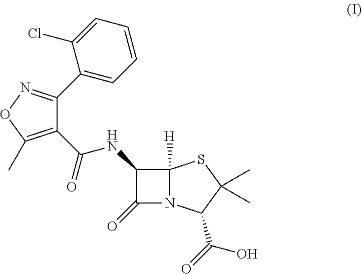 [SMILES: Cc1onc(-c2ccccc2Cl)c1C(=O)N[C@@H]1C(=O)N2[C@@H]1SC(C)(C)[C@@H]2C(=O)O]

Among aerobic bacteria, cloxacillin is used against Streptococcus pyogenes, a usually susceptible Gram-positive aerobic bacterium, and other Gram-positive aerobic bacteria such as Staphylococcus aureus and coagulase-negative staphylococci, but some staphylococci are now showing resistance.

In terms of therapeutic uses, cloxacillin is used orally in the management of staphylococcal and/or streptococcal skin infections. By injection, cloxacillin is used in the management of conditions largely associated with staphylococcal infection such as: osteoarticular infections, endocarditis, respiratory infections, urogenital infections. Staphylococcal dermatological infections can also be treated by injection.

Cloxacillin is also known in a capsule form, for example marketed under the brand name ORBENINE 500 mg capsule (cloxacillin), and is indicated in adults and children for the treatment of mild skin infections due to susceptible staphylococci and/or streptococci. In all these therapeutic uses, cloxacillin is used and described as a bactericidal antibiotic, i.e. a “bacteria-killing” molecule. For example, Panpharma 500 mg cloxacillin is intended to be administered, after solubilization, intravenously as a curative or preventive treatment. In preventive treatment, the dosage is, for an adult without kidney or liver problems, 8 to 12 g/day, divided into 4 to 6 daily administrations. In preventive treatment, the antibiotic prophylaxis to prevent post-operative infections in surgery is generally 2 g under anesthesia, followed by injections of 1 g every 2 h. For example, cloxacillin is known and used as a bacterial killer in the treatment of osteoarticular infections, especially when the responsible germ, identified, is a methicillin-sensitive strain of Staphylococcus aureus. However, although antibiotic therapy appears to be appropriate due to the sensitivity of the strain (as determined by sensitivity tests to conventional antibiotics such as Etest, Vitek® or bioMérieux Vitek® 2, antibiogram), a significant treatment failure rate of more than 20% has been reported (Valour et al., BMC Infect Dis. 2014 Aug. 16; 14:44).

In the present disclosure, biofilm means any biofilm known to the skilled person formed by a microorganism, for example, a bacterial biofilm.

In the present disclosure, surface means any surface known to the skilled person on which a biofilm can form. It may be, for example, a biotic or abiotic surface.

In the present disclosure, biotic surface means any biotic surface known to the skilled person. It may be, for example, a biotic surface selected from the group comprising skin, mucous membranes, teeth, any surface of the vascular system, for example blood vessels, walls of the endocardium, any surface of the lymphatic system, any surface of the digestive system, any surface of the respiratory system, for example walls of the bronchopulmonary system, any surface of the ear, nose and throat sphere.

In the present disclosure, the biotic surface may be a healthy surface and/or have at least one lesion and/or infection. For example, it may be a biotic surface comprising a wound, an infected wound, a healing wound, a sutured wound, a scar, a burn.

In the present disclosure, wound means any wound known to the skilled person. This may be a superficial wound, for example a wound of the epidermis and/or dermis, for example less than 5 cm in size. It can also be a deep wound, for example a wound of the epidermis and dermis with exposure of subcutaneous tissue. It can also be bums, bedsores. It can also be any wound that is likely to become infected, for example any wound that is likely to become infected because infection delays or compromises wound healing.

In the present disclosure, infection means any infection known to the skilled person for which biofilms are likely to form. Examples of such infections are selected from the group comprising osteoarticular infections, diabetic foot infections, skin wound infections such as burns and bedsores, infections of the respiratory system, for example bronchopulmonary system, ear, nose and throat infections, infections of the cardiovascular system, infections of the digestive system.

In the present disclosure, abiotic surface means any abiotic surface known to the skilled person. It may also include any device surface known to the skilled person on which bacteria may form a biofilm. For example, it may be the surface of a medical device such as a catheter, a needle, an implantable chamber catheter (or Porth-a-Cath), a valve, for example a heart valve, a prosthesis, for example a j oint prosthesis, a ligament prosthesis, a dental implant, urinary tract prosthesis, peritoneal membrane, peritoneal dialysis catheters, synthetic vascular grafts, stents, internal attachment devices, percutaneous and/or tracheal sutures, ventilation tubes.

It may be for examples any surface in medical emergency rooms, medical treatment rooms, operating rooms, operating theatres, intensive care unit (ICU) wards/rooms, infectious disease unit wards/rooms, oncology unit wards/rooms and/or other units receiving severely immunocompromised patients, isolation wards/rooms, aircraft cabins. It may also include any area of laboratory, biological laboratory, bioanalytical laboratory, incubation room and other enclosed volume of any kind in which biofilm is likely to form.

In the present disclosure, the abiotic surface may be formed by any material known to the skilled person. This may be, for example, a biocompatible or non-biocompatible material. For example, the material may be any biocompatible material known to the skilled person, for example a prosthesis, for example an osteoarticular prosthesis, a heart valve, a dental implant. For example, the material can be any material used in pipes or any container allowing the passage of a fluid, for example in production units of the food, pharmaceutical industry.

In the present disclosure, the abiotic surface can be for example a metallic surface, a surface formed by an alloy, a polymeric surface. For example, the metal may be any metal known to the skilled person, for example a metal selected from the group consisting of titanium, copper, iron, aluminum, nickel, tungsten, silver, gold, palladium, vanadium, molybdenum. For example, the alloy may be any alloy known to the skilled person, for example an alloy selected from the group consisting of steel, brass, copper-nickel, copper-palladium, silver-gold, silver-palladium, molybdenum-vanadium, molybdenum-tungsten. For example, the polymer can be any polymer known to the skilled person that can constitute and/or cover a surface. For example, it may be a polymer selected from the group comprising polytetrafluoroethylene (PTFE), polysiloxanes, polyurethanes, functionalized polymers.

In the present disclosure, the use of cloxacillin to inhibit bacterial biofilm formation on a surface may be performed at a dose from 25 to 100 mg/kg individual, preferentially 25 to 50 mg/kg individual.

Advantageously, when cloxacillin is used at a dose from 25 to 100 mg/kg individual, preferentially from 25 to 50 mg/kg individual, it allows to obtain serum cloxacillin concentrations of 4 to 16 mg/L, preferentially at serum concentrations from 4 to 8 mg/L.

In the present disclosure, the concentration of cloxacillin used may be a function of surface area.

According to the disclosure, when the surface is an abiotic surface, cloxacillin can be used at a dose from 25 to 100 mg/kg individual, for example from 25 to 50 mg/kg, and/or at a concentration from 4 to 16 mg/L, preferentially from 4 to 8 mg/L.

According to the disclosure, when the surface is a biotic surface, cloxacillin can be used at a dose from 25 to 100 mg/kg individual, for example from 25 to 50 mg/kg individual.

Advantageously, when cloxacillin is applied directly to a surface, for example biotic or abiotic, cloxacillin can be used at a concentration from 4 to 16 mg/L, preferentially from 4 to 8 mg/L.

As mentioned above, the inventors have surprisingly demonstrated that cloxacillin allows, in particular and in unusual concentrations, to inhibit the formation of biofilm and in particular to inhibit the first step necessary for this formation, namely the adhesion and attachment, also called adherence and adhesion respectively, of bacteria on a surface. Thus, the inventors have clearly demonstrated that the use of cloxacillin makes it advantageous, in particular by inhibiting the adhesion step, to prevent biofilm formation.

The present disclosure also relates to the use of cloxacillin at a concentration higher than 4 mg/L ranging from 4 to 16 mg/L to prevent the formation of biofilms on a surface. The present disclosure also relates to the use of cloxacillin at a concentration higher than 25 mg/kg, for example from 25 to 100 mg/kg, for example from 25 to 50 mg/kg to prevent the formation of biofilms on a surface.

The surface can be a surface as defined above.

In the present disclosure, to “prevent biofilm formation” means to prevent the adhesion and/or attachment of microorganisms to said surface in the presence of microorganisms.

When used for the prevention of biofilm formation on a surface, cloxacillin may be brought into contact with and/or applied to said surface by any suitable process and/or device known to the skilled person. For example, the contacting and/or application can be carried out by spraying cloxacillin onto the surface, by dipping the surface in a composition comprising cloxacillin, by spraying or dipping cloxacillin in combination with a component which is supposed to act also against the formation of biofilm, such as copper. The skilled person will, on the basis of his general knowledge, be able to select and/or adapt the known application and/or contact processes depending on the surfaces.

For example, when cloxacillin is used for the prevention and/or inhibition of bacterial biofilm formation on an abiotic surface, for example a medical device, it may be applied to the external surface of the device by any process known to the skilled person. For example, contact or application can be achieved by spraying cloxacillin onto said surface, by soaking the medical device in a solution comprising cloxacillin, by spraying or dipping said solution. The solution may further comprise one or more active agents, for example a compound known to inhibit biofilm formation, for example copper.

When cloxacillin is used for the prevention and/or inhibition of bacterial biofilm formation on an abiotic surface, cloxacillin may be in any suitable form known to the skilled person. For example, cloxacillin can be used in liquid and/or powder form.

For example, when cloxacillin is used for the prevention and/or inhibition of bacterial biofilm formation on an abiotic surface, for example a medical device, it may be applied prior to the implantation and/or use of the medical device.

When cloxacillin is used for the prevention and/or inhibition of bacterial biofilm formation on a biotic surface, cloxacillin may be in any suitable form known to the skilled person. For example, cloxacillin may be used in liquid form, for example parenterally administered, and/or in ingestible form, for example administered orally, or in a form suitable for topical application, for example an ointment, cream, woven or impregnated non-woven dressing/substrate.

The skilled person understands that the term “form” as used here refers to the pharmaceutical formulation of the medicament for its practical use.

The inventors have also surprisingly demonstrated that the use of cloxacillin according to the disclosure advantageously inhibits the formation of biofilm and in particular inhibits in particular the first step indispensable for this formation, namely the deposition and attachment, also called respectively adherence and adhesion, of bacteria, for example on wounds and/or at the level of osteoarticular infections. In particular, the inventors have demonstrated this inhibition, as described in the examples, under in vivo conditions via inoculation onto a catheter implanted subcutaneously into the thigh of a mouse and by measuring the effect, under example pharmacokinetic/pharmacodynamic conditions defined according to the effects of cloxacillin on the biofilm installation on the catheter of the same clinical strains used in vitro.

The inventors have also surprisingly demonstrated that cloxacillin, administered at a therapeutic dose based on the MIC of a strain, is ineffective in forming biofilm and thus in setting up an infection. In particular, the inventors have surprisingly demonstrated that the use of antibiotics at known therapeutic doses does not prevent and/or inhibit the formation/development of a biofilm in a medical device, for example an implanted catheter.

The present disclosure therefore also relates to the use of cloxacillin at a dose higher than 25 mg/kg comprised from 25 to 100 mg/kg individual as a medicament for inhibiting the formation and/or development of a biofilm on a surface.

The present disclosure therefore also relates to the use of cloxacillin at a dose higher than 25 mg/kg, for example from 25 to 100 mg/kg, from 25 to 50 mg/kg individual as a medicament for inhibiting the formation and/or development of a biofilm on a surface.

The surface can be a surface as defined above. For example, the surface can be an abiotic surface, such as an external surface of a medical prosthesis. The surface can also be a biotic surface, for example skin, mucous membranes, teeth, any surface of the vascular system, the walls of the endocardium, any surface of the lymphatic system, any surface of the ear, nose and throat sphere.

In the present disclosure, the use of cloxacillin as a medicament according to the disclosure may be carried out simultaneously on several surfaces, for example in the same mammal. For example, the use may be carried out on a biotic and abiotic surface, for example on a surface of a prosthesis and on a surface of biological tissues surrounding said prosthesis. For example, use may be performed on at least two biotic and/or abiotic surfaces.

In the present disclosure, “inhibition of biofilm formation” means the inhibition in the presence of microorganisms, for example biofilm-forming microorganisms, of the adhesion and/or attachment thereof to said surface.

In the present disclosure, “inhibition of biofilm development on a surface” means inhibition in the presence of microorganisms, for example biofilm-forming microorganisms, of their adhesion and/or attachment to said surface.

According to the disclosure, the medicament may be intended for a mammal, for example a mammal selected from the group comprising the order Monotremata, Didelphimorphia, Paucituberculata, Microbiotheria, Notoryctemorphia, Dasyuromorphia, Peramelemorphia, Diprotodontia, Tubulidentata, Sirenia, Afrosoricida, Macroscelidea, Hyracoidea, Proboscidea, Cingulata, for example the armadillo, Pilosa, Scandentia, Dermoptera, Primates, Rodentia, Lagomorpha, Erinaceomorpha, Soricomorpha, Chiroptera, Pholidota, Carnivora, Perissodactyla, Artiodactyla and Cetacea.

It can be a human or an animal, for example. For example, it could be a farm animal, a pet, an endangered animal or any other animal.

For example, the farm animal may be selected from the group consisting of cattle, pigs, sheep, goats, camels, dogs, horses, murines. For example, the pet animal may be chosen from the group comprising canines and felines.

In the present disclosure, medicament means a medicament for human use or a veterinary medicament.

In the present disclosure, individual means a mammal, for example a mammal selected from the group consisting of the order Monotremata, Didelphimorphia, Paucituberculata, Microbiotheria, Notoryctemorphia, Dasyuromorphia, Peramelemorphia, Diprotodontia, Tubulidentata, Sirenia, Afrosoricida, Macroscelidea, Hyracoidea, Proboscidea, Cingulata, for example the armadillo, Pilosa, Scandentia, Dermoptera, Primates, Rodentia, Lagomorpha, Erinaceomorpha, Soricomorpha, Chiroptera, Pholidota, Carnivora, Perissodactyla, Artiodactyla and Cetacea.

It can be a human or an animal, for example. For example, it may be a farm animal, a pet, an endangered animal or any other animal.

For example, the farm animal may be selected from the group consisting of cattle, pigs, sheep, goats, camels, dogs, horses, murines. For example, the pet animal may be chosen from the group comprising canines and felines.

For use as a medicament, cloxacillin may be in any form that can be administered to a human or animal. Administration may be carried out directly, i.e. pure or virtually pure, or after mixing cloxacillin with a support and/or a pharmaceutically acceptable carrier.

According to the present disclosure, the medicament may be in the form of a powder, for example for injectable solution, or for oral administration to be swallowed in the form of capsules. According to the present disclosure, the medicament may be a medicament for oral administration. For example, when the medicament is a medicament for oral administration, it may be in the form of a capsule.

According to the present disclosure, the medicament may be a medicament for parenteral administration, for example intravenous administration.

According to the present disclosure, the medicament may be a medicament for topical administration, for example for skin application, for example an ointment, a cream, an impregnated bandage, a galenic patch, a spray.

According to the disclosure, when cloxacillin is used as a medicament, it may be at a dose from 25 to 100 mg/kg individual, for example from 25 to 50 mg/kg individual and/or at a concentration from 4 to 16 mg/L, for example from 4 to 8 mg/L.

According to the disclosure, when the surface is a biotic surface, cloxacillin can be used at a concentration from 4 to 16 mg/L, for example from 4 to 8 mg/L.

According to the disclosure, when the surface is a biotic surface, cloxacillin can be used at a concentration from 25 to 100 mg/kg, for example from 25 to 50 mg/kg individual, and/or at a concentration from 4 to 16 mg/L, for example from 4 to 8 mg/L.

According to the disclosure, the use of cloxacillin as a medicament can be carried out at a concentration such that its bioavailability at the surface level can be comprised from 4 to 16 mg/L.

The inventors have surprisingly demonstrated that the use of cloxacillin at particular amounts/concentrations has the advantage of inhibiting the formation and/or development of a biofilm on the surface of medical devices, including medical devices implanted in a mammal. In particular the inventors have surprisingly demonstrated that the use of cloxacillin according to the disclosure makes it possible advantageously to inhibit the formation and/or development of a biofilm on the surface, in particular of an implanted catheter, whereas the use of cloxacillin at a usual therapeutic dose, i.e. that used for the treatment of bacterial infections, does not make it possible to prevent the implantation and/or the formation of the biofilm nor to inhibit its development.

In other words, the use of cloxacillin according to the disclosure advantageously prevents/inhibits biofilm formation and also the installation of a bacterial infection/colony contrary to known uses.

The inventors have thus demonstrated that the use according to the disclosure makes it advantageous to prevent bacterial tolerance and/or resistance and/or multi-resistance related to the formation and/or development of a biofilm on a surface.

The inventors have also demonstrated that the use according to the disclosure is advantageous in preventing bacterial tolerance related to the formation and/or development of a biofilm on a surface.

The inventors have also demonstrated that the use according to the disclosure is advantageous in preventing bacterial resistance related to the formation and/or development of a biofilm on a surface.

The present disclosure therefore also relates to cloxacillin for use as a medicament for preventing multi-bacterial resistance related to the formation and/or development of a biofilm on a surface.

The present disclosure is therefore also intended for the use of cloxacillin at a dose higher than 25 mg/kg individual, for example from 25 to 100 mg/kg individual and/or at a concentration of between 4 and 16 mg/L, for example from 4 to 8 mg/L, as a medicament for preventing multi-bacterial resistance linked to the formation and/or development of a biofilm on a surface.

In other words, the present disclosure is also intended for the use of cloxacillin at a dose higher than 25 mg/kg individual, for example from 25 to 100 mg/kg, from 25 to 50 mg/kg individual as a medicament to prevent multi-bacterial resistance linked to the formation and/or development of a biofilm on a surface.

Advantageously, when cloxacillin is used at a dose higher than 25 mg/kg individual, for example from 25 and 100 mg/kg individual as a medicament to prevent multi-bacterial resistance linked to the formation and/or development of a biofilm on a surface, it makes it possible to obtain a serum concentration of cloxacillin from 4 and 16 mg/L which advantageously makes it possible to prevent multi-bacterial resistance linked to the formation and/or development of a biofilm on a surface.

In the present disclosure, multi-bacterial resistance is defined as the ability of bacteria to resist more than one antibiotic treatment, in particular the ability of bacteria to resist two or more antibiotic treatments.

The present disclosure therefore also relates to cloxacillin for use as a medicament for preventing bacterial tolerance related to the formation and/or development of a biofilm on a surface.

The present disclosure is also intended for the use of cloxacillin at a dose higher than 25 mg/kg individual, for example from 25 to 100 mg/kg individual and/or at a concentration from 4 and 16 mg/L, for example from 4 to 8 mg/L, as a medicament for preventing bacterial tolerance related to the formation and/or development of a biofilm on a surface.

In other words, the disclosure invention is also intended for the use of cloxacillin at a dose higher than 25 mg/kg individual, for example from 25 to 100 mg/kg, from 25 to 50 mg/kg individual as a medicament to prevent bacterial tolerance related to the formation and/or development of a biofilm on a surface.

Advantageously, when cloxacillin is used at a dose higher than 25 mg/kg individual, for example between 25 and 100 mg/kg individual as a medicament to prevent bacterial tolerance linked to the formation and/or development of a biofilm on a surface, it makes it possible to obtain a serum concentration of cloxacillin of between 4 and 16 mg/L, making it possible advantageously to prevent bacterial tolerance linked to the formation and/or development of a biofilm on a surface.

In the present disclosure, bacterial tolerance means the ability of bacteria to resist naturally, i.e. without the identification of mutation(s) or gene(s) conferring resistance(s), one or more antibiotic treatment(s).

The present disclosure also relates to cloxacillin for use as a medicament for preventing bacterial resistance related to the formation and/or development of a biofilm on a surface.

The present disclosure is also intended for the use of cloxacillin at a dose higher than 25 mg/kg individual, for example from 25 to 100 mg/kg individual and/or at a concentration from 4 and 16 mg/L, for example from 4 to 8 mg/L, as a medicament to prevent bacterial resistance related to the formation and/or development of a biofilm on a surface.

In other words, the present disclosure is also intended for the use of cloxacillin at a dose higher than 25 mg/kg individual, for example from 25 to 100 mg/kg, from 25 to 50 mg/kg individual as a medicament to prevent bacterial resistance related to the formation and/or development of a biofilm on a surface.

Advantageously, when cloxacillin is used at a dose higher than 25 mg/kg individual, for example from 25 and 100 mg/kg individual as a medicament to prevent bacterial resistance linked to the formation and/or development of a biofilm on a surface, it makes it possible to obtain a serum concentration of cloxacillin of between 4 and 16 mg/L which advantageously makes it possible to prevent bacterial resistance linked to the formation and/or development of a biofilm on a surface.

In the present disclosure, bacterial resistance means the ability of bacteria that are mutant or have acquired one or more resistance genes to resist one or more antibiotic treatments. In particular, mutation and/or acquisition of the resistance gene enables the bacterium to resist one or more antibiotic treatment(s).

The conditions, amounts and routes of administration of the cloxacillin used according to the disclosure may be as described herein, for example above. The skilled person, by virtue of this general knowledge, will be able to adapt the conditions and forms used according to the surface.

Advantageously, the inventors have demonstrated that the use according to the disclosure makes it possible to inhibit or prevent biofilm formation on a surface and thus a possible tolerance (also called recalcitrance) and/or resistance to antibiotics, in particular due to the presence of biofilm. Thus, use according to the present disclosure makes it possible both to inhibit or prevent biofilm formation and possibly to treat the infection.

The inventors have also demonstrated that the use according to the disclosure of cloxacillin on biotic surfaces, for example on skin, for example on skin wounds, has the advantage of preventing and/or inhibiting the formation of biofilm. Advantageously, the use according to the disclosure makes it possible to prevent and/or inhibit further infection and/or superinfection by biofilm-forming bacteria, for example in wounds.

The present disclosure is therefore also intended for the use of cloxacillin at a concentration higher than 25 mg/kg individual, for example from 25 to 100 mg/kg individual and/or at a concentration between 4 and 16 mg/L, for example from 4 to 8 mg/L, as a medicament for preventing and/or inhibiting further infection and/or superinfection by bacteria forming biofilms of an infected biotic surface.

In other words, the present disclosure is also intended for the use of cloxacillin at a dose higher than 25 mg/kg individual, for example from 25 to 100 mg/kg, from 25 to 50 mg/kg individual as a medicament for preventing and/or inhibiting further infection and/or superinfection by bacteria forming biofilms of an infected biotic surface.

In the present disclosure, “preventing and/or inhibiting further infection and/or superinfection by bacteria” means inhibiting in the presence of microorganisms, for example biofilm-forming microorganisms, the adhesion and/or attachment thereof to said infected surface.

In the present disclosure, an infected biotic surface is any biotic surface known to the skilled person that may have a bacterial infection.

In the present disclosure, bacterial infection means any bacterial infection known to the skilled person that may affect a biotic or abiotic surface.

Examples of bacterial infections; for example of biotic surfaces, for example of the skin can be abscesses, folliculitis, boils, furunculosis, anthrax, panaris, phlegmon, pearl, intertrigo, stye, erysipelas, impetigo, prurigo.

According to the disclosure, when cloxacillin is used as a medicament for preventing and/or inhibiting further infection and/or superinfection by bacteria forming biofilms of an infected biotic surface, it can be used at a dose of 25 to 100 mg/kg individual, for example from 25 to 50 mg/kg individual and/or at a concentration between 4 and 16 mg/L, for example 4 to 8 mg/L.

In other words, according to the disclosure, when cloxacillin is used as a medicament for preventing and/or inhibiting further infection and/or superinfection by bacteria forming biofilms of an infected biotic surface, it can be used at a dose from 25 to 100 mg/kg individual, for example from 25 to 50 mg/kg individual.

Advantageously, when cloxacillin is used at a dose greater than 25 mg/kg individual, for example from 25 to 100 mg/kg individual as a medicament to prevent and/or inhibit further infection and/or superinfection by bacteria forming biofilms of an infected biotic surface, it allows a serum concentration of cloxacillin to be obtained of between 4 and 16 mg/L, for example 4 to 8 mg/L, making it advantageous to prevent multi-bacterial resistance linked to the formation and/or development of a biofilm on a surface.

Depending on when cloxacillin is used as a medicament to prevent and/or inhibit further infection and/or superinfection by bacteria forming biofilms of an infected biotic surface, it may be used at a concentration of 25 to 100 mg/kg individual, for example from 25 to 50 mg/kg individual and/or at a concentration between 4 and 16 mg/L, for example from 4 to 8 mg/L.

In other words, according to the disclosure, when cloxacillin is used as a medicament for preventing and/or inhibiting further infection and/or superinfection of an infected biotic surface with biofilm-forming bacteria, it can be used at a dose from 25 to 100 mg/kg individual, for example from 25 to 50 mg/kg individual.

Advantageously, when cloxacillin is used at a dose greater than 25 mg/kg individual, for example from 25 to 100 mg/kg individual as a medicament to prevent and/or inhibit further infection and/or superinfection by bacteria forming biofilms of an infected biotic surface, it allows a serum concentration of cloxacillin to be obtained of between 4 and 16 mg/L, for example from 4 to 8 mg/L, making it advantageous to prevent multi-bacterial resistance linked to the formation and/or development of a biofilm on a surface.

According to the disclosure, the use of cloxacillin as a medicament can be carried out at a concentration such that its bioavailability at the surface can be from 4 to 16 mg/L.

For use as a medicament for preventing and/or inhibiting further infection and/or superinfection by bacteria forming biofilms of an infected biotic surface, cloxacillin may be in any suitable form known to the skilled person. This may be a form as mentioned above.

Thanks to this general knowledge, the skilled person will be able to adapt the conditions and forms used according to the surface.

The present disclosure also relates to a pharmaceutical composition comprising cloxacillin at a dose of more than 0.4%, for example from 0.4% to 1.6% in weight percentage based on the total weight of the composition intended to be used as a medicament for the prevention of biofilm formation on a surface.

The present disclosure also relates to a pharmaceutical composition comprising cloxacillin at a dose higher than 4 mg/L, for example from 4 to 16 mg/L, for use as a medicament for the prevention of biofilm formation on a surface.

The use of the composition according to the disclosure may be carried out on any surface known to the skilled person. This may be, for example, a biotic or abiotic surface as defined above. It may be, for example, a biotic or abiotic surface as defined above. It may be, for example one or more surfaces as defined above.

In the present disclosure, when using a pharmaceutical composition comprising cloxacillin as a medicament for the prevention of surface bacterial biofilm formation, the cloxacillin concentration/amount may be greater than 0.4%, for example from 0.4% to 1.6% by weight percent based on the total weight of the composition.

In the present disclosure, when using a pharmaceutical composition comprising cloxacillin as a medicament for the prevention of surface bacterial biofilm formation, the cloxacillin concentration/amount may be greater than 4 mg/L for example from 4 to 16 mg/L.

The conditions, amounts and routes of administration of the composition according to the disclosure may be as described herein, for example above. The skilled person, by virtue of his general knowledge, will know how to adapt the conditions and means of administration of the composition.

In the present disclosure, the pharmaceutical compositions of the present disclosure may further comprise a pharmaceutically acceptable carrier, an adjuvant or a vehicle.

In the present disclosure, a pharmaceutically acceptable carrier, adjuvant or vehicle includes any solvent, diluent or other liquid carrier, dispersion or suspension aid, surfactant, isotonic agent, thickening agent or emulsifier, preservative, solid binder, lubricant and the like, suitable for the particular dosage form desired. Remington Pharmaceutical Sciences, Sixteenth Edition, E. W. Martin (Mack Publishing Co., Easton, Pa., 1980) describes various carriers used in the formulation of pharmaceutically acceptable compositions and techniques known for their preparation. Unless a conventional carrier medium would prove incompatible with the compounds according to the disclosure, for example by producing any undesirable biological effect or by interacting in a deleterious manner with any other component(s) of the pharmaceutically acceptable composition, its use is considered to fall within the scope of the present disclosure. Some examples of constituents that may serve as pharmaceutically acceptable carriers include, but are not limited to, ion exchangers, alumina, aluminum stearate, lecithin, serum proteins such as human serum albumin, buffer substances such as phosphates, glycine, sorbic acid, or potassium sorbate, mixtures of partial glycerides of saturated vegetable fatty acids, water, salts or electrolytes such as protamine sulfate, disodium phosphate, potassium hydrogen phosphate, sodium chloride, zinc salts, colloidal silica, magnesium trisilicate, polyvinylpyrrolidone, polyacrylates, waxes, polyethylene polyoxypropylene polymers, sugars such as lactose, glucose and sucrose; starches such as corn and potato starch; cellulose and its derivatives such as sodium carboxymethylcellulose, ethylcellulose and cellulose acetate; tragacanth powder; malt; gelatin; talc; excipients such as cocoa butter and waxes for suppositories; oils such as peanut oil, cottonseed oil; safflower oil; sesame oil; olive oil; corn oil and soybean oil; glycols; such propylene glycol or polyethylene glycol; esters such as ethyl oleate and ethyl laurate; agar; agents such as magnesium hydroxide and buffered aluminum hydroxide; alginic acid; isotonic salt solution; Ringer's solution; ethyl alcohol, and phosphate buffer solutions, as well as other compatible non-toxic lubricants such as sodium lauryl sulfate and magnesium stearate, as well as coloring agents, mold release agents, coating agents, sweetening, flavoring and perfuming agents, preservatives and antioxidants may also be present in the composition, according to the pharmaceutical formulator's judgement.

The pharmaceutically acceptable compositions of the present disclosure may be administered to humans and other animals orally, rectally, parenterally, intracisternally, intravaginally, intraperitoneally, topically (such as by powders, ointments or drops), orally, as an oral or nasal spray, or similarly, depending on the severity of the infection to be treated.

Liquid dosage forms for oral administration include, but are not limited to, pharmaceutically acceptable emulsions, microemulsions, solutions, suspensions, syrups and elixirs. In addition to active compounds, liquid dosage forms may contain inert diluents commonly used in the art such as, for example, water or other solvents, agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, peanut, corn, germ, olive, and sesame seeds), glycerol, tetrahydrofurfuryl alcohol, polyethylene glycols and sorbitan fatty acid esters and mixtures thereof. In addition to inert diluents, oral compositions may also include adjuvants such as wetting agents, emulsifying and suspending agents, sweeteners, flavorings and perfuming agents.

Solid dosage forms for oral administration include capsules, tablets, pills, powders and granules. In such solid dosage forms, the active compound is mixed with at least one inert carrier, excipient or pharmaceutically acceptable carrier such as sodium citrate or dicalcium phosphate and/or a) fillers or diluents such as starches, lactose, sucrose, glucose, mannitol and silicic acid, b) binders such as, for example, carboxymethylcellulose, alginate, gelatin, polyvinylpyrrolidone, sucrose and gum arabic, c) humectants such as glycerol, d) disintegrants such as agar-agar, calcium carbonate, potato or cassava starch, alginic acid, certain silicates and sodium carbonate, e) absorption accelerators such as quaternary ammonium compounds, f) wetting agents such as, for example cetyl alcohol and glycerol monostearate, g) absorbents such as kaolin, talc and bentonite clay, and h) lubricants such as calcium stearate, magnesium stearate, solid polyethylene glycols, sodium lauryl sulfate and mixtures thereof. In the case of capsules, tablets and pills, the dosage form may also include buffering agents.

Solid compositions of a similar type may also be used as fillers in soft or hard gelatin capsules using excipients such as lactose or milk sugar as well as high molecular weight polyethylene glycols and the like. Solid dosage forms of tablets, coated tablets, capsules, pills and granules can be prepared with coatings and shells such as enteric coatings and other coatings well known in the pharmaceutical art of formulation. They may optionally contain opacifying agents and may also be of such a composition that they release the active agent(s) only, or preferentially, in a certain part of the intestinal tract, optionally in a delayed manner. Examples of coating compositions that may be used include polymeric substances and waxes. Solid compositions of a similar type may also be used as fillers in soft and hard gelatin capsules using excipients such as lactose or milk sugar as well as high molecular weight polyethylene glycols and the like.

In the present disclosure, the uses and/or compositions according to the disclosure may be combined with conventional antibacterial agents, such as antibiotics, for example bactericides and/or bacteriostats. For example, conventional antibacterial agents may be used or administered prior to, or simultaneously with, the use and/or composition according to the disclosure.

In the present disclosure, the uses and/or compositions according to the disclosure may be combined with conventional antifungal agents, for example commercially available antifungal agents, for example miconazole. For example, conventional antifungal agents may be used or administered prior to, or simultaneously with, the use and/or composition according to the disclosure.

For example, cloxacillin can be used according to the disclosure and/or the pharmaceutical composition can be used according to the disclosure in combination with at least one of the antibiotics selected from the group consisting of aminoglycosides, fluoroquinolones, macrolides, novobiocin, rifampicin, oxazolidinones, fusidic acid, mupirocin, pleuromutilins, daptomycin, vancomycin, tetracyclines, sulfonamides, chloramphenicol, trimethoprim, fosfomycin, cycloserine and polymyxin.

Other advantages may also appear to the skilled person upon reading the examples below, illustrated by the appended figures, which are provided by way of illustration.

### EXAMPLES

### Example 1

### In Vitro Demonstration of Cloxacillin as an Inhibitor of Biofilm Formation

In this example, an Antibiofilmogram was performed to demonstrate cloxacillin inhibition of biofilm formation and to determine cloxacillin doses effective against biofilm formation in a polystyrene microplate well. This example clearly demonstrates that the minimum inhibitory concentrations (MICb) for biofilm formation are different from the minimum inhibitory concentrations (MICs) that define antibiotic susceptibility to the antibiotic in relation to its bactericidal activity. These MICs may differ between two strains while the MICs for the same strains are close (0.125 and 0.25 μg/mL, or mg/L) and on the other hand may be very different from the MICs.

To determine and measure the effect of cloxacillin on biofilm formation, specifically bacterial adhesion, the first step in biofilm formation, Staphylococcus aureus strains S14 and S39 were incubated in the wells of a 96-well microplate in the presence of varying concentrations of cloxacillin. The concentrations tested ranged from 0.25 μg/mL to 32 μg/mL cloxacillin. This test is called, by BioFilm Control, an Antibiofilmogram®. The inoculum of bacteria was 4·10⁶ bacteria/mL, in 200 μL brain heart infusion (BHI) medium, in the presence of magnetic microbeads (Toner 4, product code TON004) at a concentration of 2 μL per 200 μL, i.e. 10 μL/mL).

The microplate was incubated for 4 hat 37° C., then covered with 120 μL of contrast liquid (Contrast liquid; product code LIC001) and placed for 1 minute on a magnet block (product code BKTMB) designed so that each mini-magnet is centered with respect to the 96 wells. The microplate was then placed on a scanner and the image was acquired and analyzed by the BFCE3 software which quantifies the aggregation of the magnetic microbeads in the center of the wells and provides a biofilm formation index (BFI) value. A BFI value of 20, or close to 20 corresponds to maximum aggregation, therefore no biofilm formation, and therefore cloxacillin anti-adhesion efficacy. Conversely, a BFI of 0 corresponds to a total immobility of the microbeads completely blocked by the adhering bacteria. All intermediate values, allowing a quantitative measurement of the efficacy of cloxacillin are possible.

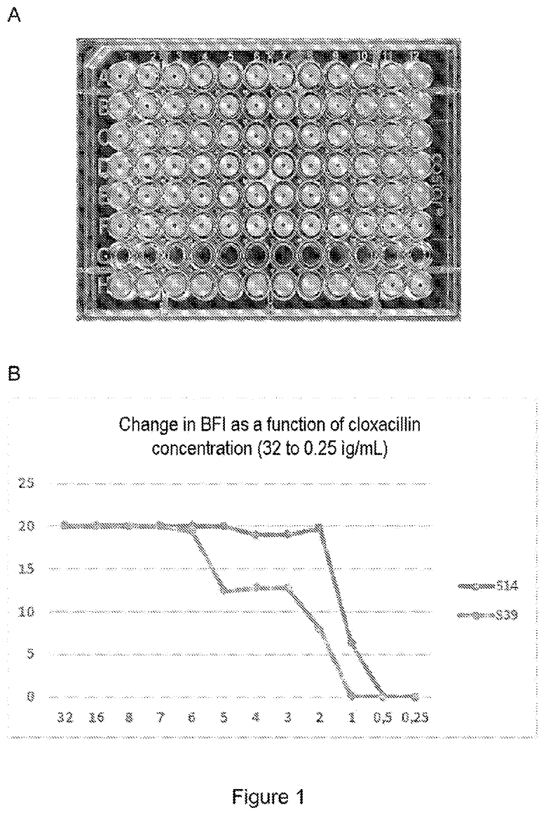

FIG. 1A is a scanned image of the plate after magnetization and represents the raw result of the Antibiofilmogram® of strain S14 and FIG. 1B is a diagram showing the change in BFI (ordinate) as a function of the cloxacillin concentration in pg/mL.

In FIG. 1A, lines E and A correspond respectively to strains S14 and S39 grown in the presence of varying concentrations of cloxacillin. Lines B, C and D correspond to other strains not mentioned here. Line F corresponds to controls in the absence of strain (normal presence of a spot) and wells H1 and H2 correspond to strain S39 alone, and wells H9 and H10 correspond to strain S39. The strains are tested at the following concentrations, starting from column 1 to column 12): 32, 16, 8, 7, 6, 5, 4, 3, 2, 1, 05, 0.25 82 g/mL.

FIG. 1B shows the curves corresponding to the BFIs measured in the wells in FIG. 1A. As shown for strain S14 a concentration of 2 pg/mL allows total mobility of the 2 5 microbeads and for strain S39 a concentration of 6 pg/mL allows total mobility of the microbeads, with a steepness of the curve for concentrations between 3 and 5 μg/mL. As shown in FIG. 1 B, the MICb of cloxacillin is 6 μg/mL (or 4-6 μg/mL) for strain S39 and 2 μg/mL for Staphylococcus aureus strain S14.

In the present example, the antibiotic film effect of cloxacillin was also demonstrated on 26 clinical strains of Staphylococcus aureus. The experimental procedure and protocol were as described above.

The determination of each minimum inhibitory concentration (MIC) of cloxacillin was measured conventionally by measuring the growth of each strain in 96-well microplates in the presence of concentrations of antibiotic by absorbance.

The MIC was measured in Mueller-Hinton medium (MH; reference medium for antibiotic susceptibility testing) and brain heart infusion (BHI) medium.

The measurement of the MICb (determination of the biofilm minimum inhibitory concentration), using the BioFilm Ring ^{Test}® was performed in BHI medium. The conversion in mg/kg. The results are summarized in Table 1 according to the strains tested.

| | MH MIC | BHI MIC | MIC b | mg/kg (MIC b |
|---|---|---|---|---|
| STRAINS | (μg/ml) | (μg/ml) | (μg/ml) | equivalent) |
| S02 | 0.125 | 0.25 | 2 | 15 |
| S04 | 0.25 | 0.5 | 4 | 25 |
| S08 | 0.25 | 0.5 | 2 | 15 |
| S11 | 0.5 | 0.5 | 4 | 25 |
| S14 | 0.25 | 0.25 | 2 | 15 |
| S16 | 0.25 | 0.5 | 4 | 25 |
| S19 | 0.5 | 0.5 | 2 | 15 |
| S21 | 0.25 | 0.5 | 4 | 25 |
| S22 | 0.5 | 0.5 | 4 | 25 |
| S26 | 0.25 | 0.25 | 4 | 25 |
| S27 | 0.25 | 0.25 | 4 | 25 |
| S31 | 0.25 | 0.5 | 4 | 25 |
| S34 | 0.5 | 0.5 | 8 | 50 |
| S38 | 0.5 | 0.5 | 16 | 95 |
| S39 | 0.5 | 0.125 | 8 | 50 |
| S40 | 0.5 | 0.5 | 4 | 25 |
| S43 | 0.25 | 0.25 | 4 | 25 |
| S44 | 0.25 | 0.5 | 4 | 25 |
| S46 | 0.25 | 0.125 | 2 | 15 |
| S47 | 0.25 | 0.25 | 2 | 15 |
| S51 | 0.25 | 0.25 | 4 | 25 |
| S58 | 0.25 | 0.25 | 16 | 95 |
| S62 | 0.25 | 0.5 | 8 | 50 |
| S63 | 0.25 | 0.25 | 8 | 50 |
| S74 | 0.25 | 0.25 | 4 | 25 |
| S83 | 0.125 | 0.125 | 16 | 95 |

The MICs in MH and BHI media were very similar, showing that the culture medium in which the bacteria were grown for the cloxacillin sensitivity test was without effect.

MICb values were consistently higher than MIC values, and varied between strains. Table 1 clearly demonstrates that obtaining an MIC value does not predict the MICb. In particular, the above results clearly demonstrate that the cloxacillin concentration for the antibiofilm effect is independent of the MIC and therefore does not predict any corresponding value. For example, strains S02 and S83 have MICs, in MH medium, of 0.125 μg/mL while their MICb were 2 and 16 μg/mL respectively. Conversely, strains S38, S58 and S83 have MICb of 16 μg/mL and have MICs of 0.5, 0.25 and 0.125 μg/mL, respectively.

This example shows that, on a panel of 26 strains, the mean MICb was 5.5 μg/mL with a standard deviation of 4.2 μg/mL. Only 7 strains out of the 26 have an MICb value higher than this mean (with MICb values of 8 or 16 μg/mL, and only 3 strains have an MICb value higher than the mean +standard deviation (9.7 μg/mL), with an MICb value of 16 μg/mL. The administration of cloxacillin, expressed in mg/kg, leading to a minimum serum concentration between 2 administrations, expressed in μg/mL or mg/L, greater than 8 or 16 μg/mL is likely to be effective against all Staphylococcus aureus as well as other microorganisms with a similar MICb value, i.e. within the range of values defined by the standard deviation, for example less than 9.7 μg/mL. In order to prevent biofilm formation and/or to obtain an antibiotic film effect for bacteria with an MICb of 8 mg/L, cloxacillin may be used at a dose of 49 mg/kg individual, the use at this dose providing an adequate serum concentration. In order to prevent biofilm formation and/or to achieve an antibiotic film effect for bacteria with an MICb of 16 mg/L, cloxacillin may be used at a dose of 95 mg/kg per individual, at which dose an appropriate serum concentration is obtained.

As demonstrated in this example the use of cloxacillin as a medicament to inhibit the formation and/or development of biofilm on a surface is performed at concentrations and/or doses very different from those known to have an antibiotic effect. For example, for a bacterium having an MICb of 8 mg/L, the administration of cloxacillin at a dose, for example of 49 mg/kg of said subject advantageously inhibits the formation and/or development of biofilm by said bacterium.

This example clearly demonstrates that the use of cloxacillin at concentrations greater than or equal to 4 mg/L or from 4 mg/L to 16 mg/L, corresponding to individual/patient administrations of 25 to 100 mg/kg, allows inhibition of biofilm formation, particularly by the majority of strains of Staphylococcus aureus. This example therefore also clearly demonstrates that the concentrations useful for inhibiting biofilm formation are very different and distinct from the usual therapeutic concentrations.

### Example 2

### Confocal Microscopic In Vitro Demonstration of Inhibition of Bacterial Adhesion to a Surface by Cloxacillin

Example 2 confirms the interpretations of the Antibiofilmogram by confocal microscopy. It is clearly shown that the antibiotic cloxacillin, used at the MIC dose (dose defined as sufficient for the bactericidal activity of cloxacillin on the strain under consideration) namely strain S14 and S39 is not active on the formation of biofilm on plastic. However, at the MICb dose (defined as effective in preventing biofilm formation), the strain did not adhere to the surface. These results were obtained for strains S14 and S39 which have MICs very close to each other and characterize the strains as cloxacillin-sensitive.

In other words, this example clearly demonstrates that cloxacillin can inhibit biofilm formation regardless of its known antibiotic effect. Six-well polystyrene microplates of the same nature and with exactly the same properties as the microplates used in Example 1 (Antibiofilmogram®), i.e. Costar 6-well microplates, product code 3736. The 6-well microplates were used to obtain a sufficient surface area of biofilm formation for confocal microscopy observation. The concentrations corresponding to the MIC and MICb of the strains were tested. At the same time, controls in the absence of antibiotics, strains S14 and S39 were performed. After incubation for 4 h at 37° C. (similar to the conditions used in Example 1 (Antibiofilmogram®), the bottoms of the microplates were cut out and assembled with a glass slide in order to be able to proceed to the observation under confocal microscopy. The observation conditions were as follows: Objective: ×40; Zoom: ×2; Format: 2048×2048 pixels; Laser power; Zmax and Zmin: number of images in the stack; Size between each image: 0.42 μm. “Live/Dead” staining was performed. SYTO9 stains live (and dead) bacteria, while propidium iodide specifically stains dead bacteria. The result was obtained with an analysis of 2880 images (“Slices”).

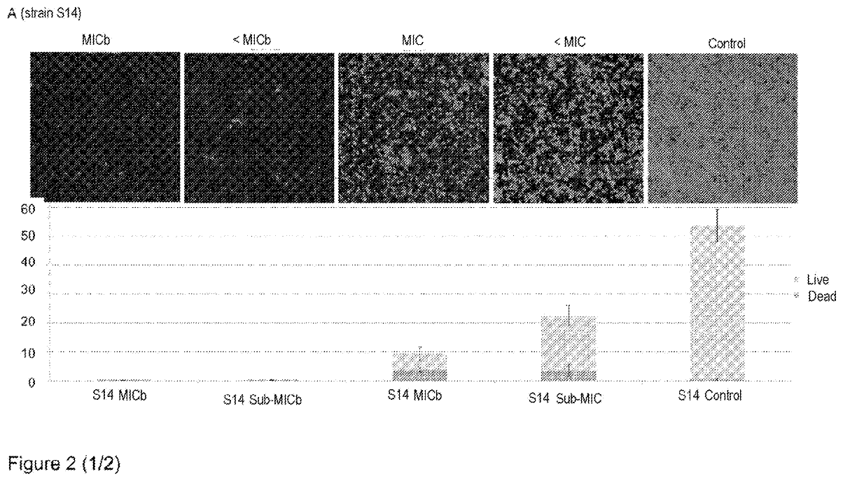

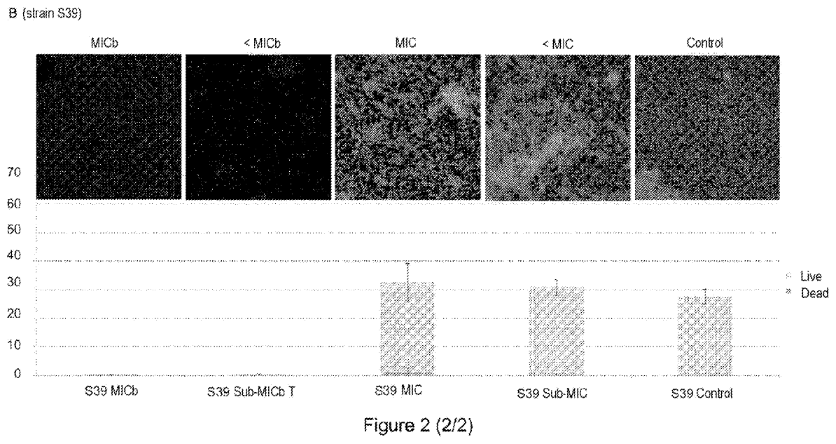

The results are shown in FIG. 2. The percentage of biofilm covering the bottom surface of the well bottom as a percentage of surface cover is shown on the vertical axis. In FIG. 2A, the density of S14 bacteria adhered to the bottom of the wells was shown and analyzed. As shown in FIG. 2A in the absence of antibiotic and/or at concentrations below the MIC, namely 0.5 MIC (sub-MIC) the antibiotic had no effect on biofilm formation. In addition, this example also demonstrates that the antibiotic, at the MIC dose, allows a decrease in bacterial adhesion but does not inhibit it. In particular, from 10% to more than 20% of the bottom surface of the well was covered after 4 h of incubation, which is excessive to consider a use in a therapeutic treatment at this dose.

The MICb (2 μg/mL) and sub-MICb (1 μg/mL; lower than MICb, but higher than MIC) doses were able to completely inhibit the adhesion of S. aureus strain S14. Indeed, as shown in FIG. 2A, incubation at a concentration equal to the MICb and sub-MICb doses inhibited the adhesion of bacteria, particularly S. aureus strain S14, and thus the formation of biofilm. On the other hand, the MIC (0.25 μg/mL) and sub-MIC (0.125 μg/mL) doses did not inhibit the adhesion of the S14 strain since 10% of the surface is covered by the bacteria at the MIC dose.

FIG. 2B shows the results obtained with S. aureus strain S39. In particular, FIG. 2B represents the density of S39 bacteria adhered to the polystyrene and includes the corresponding analysis. As demonstrated, compared to the control (control) which did not include an antibiotic, the sub-MIC (1/2 MIC) and MIC (0.125 μg/mL) doses had no effect on bacterial adhesion. The results obtained are similar to the effect of the control condition (without antibiotic).

The MICb (6 μg/mL) and sub-MICb (3 μg/mL; lower than MICb, but higher than MIC) doses were able to completely inhibit the adhesion of S. aureus strain S39. Indeed, as shown in FIG. 2B, incubation at a concentration equal to the MICb and sub-MICb doses inhibited the adhesion of bacteria, particularly S. aureus strain S39, and thus the formation of biofilm.

### Example 3

### In Vivo Demonstration of Inhibition of Biofilm Formation/Adhesion of Bacteria on a Surface by Cloxacillin

In this example, the effect of cloxacillin was observed on the infection of an implanted catheter in an animal model.

This example demonstrates and confirms in an animal model of infection that cloxacillin, used at doses related to the MICb, inhibited the adhesion of bacteria to the catheter and can therefore be used to prevent infection of a catheter (simulating a prosthesis).

Two Staphylococcus aureus strains, S14 and S39, were used. They are characterized, for cloxacillin, by minimum inhibitory concentrations (MICs) of 0.125 μg/mL for S14 and 0.25 μg/mL for S39, as well as minimum inhibitory concentrations for biofilm formation (MICb) of 2 μg/mL for S14 and 4 μg/mL for S39. A cloxacillin concentration that may be underestimated for the MICb was used in this in vivo model.

The inoculum size tested was 7 log CFU/mouse (8.3 log CFU/mL). The strains were isolated on Chapman agar medium and cultured at 37° C. for 18 h. For each strain, an isolated colony was resuspended in 9 mL of BHI medium with shaking for 6 h at 37° C. and the resulting broth was flooded on MH agar medium (beef extract 2 g/L; casein acid hydrolysate 17.5 g/L; starch 1.5 g/L; agar 17 g/L) placed at 37° C. for 18 h. The plated culture was then scraped in 10 mL of sterile saline (in the presence of glass beads to avoid the formation of aggregates) and vortexed for 5 seconds at maximum speed. Successive dilutions were performed to measure the optical density at a wavelength of 595 nm (OD595nm) (Eppendorf Spectrophotometer) and the bacterial load after culturing the dilutions on MH agar medium was determined. It was considered that an OD =0.250 resulted in an inoculum of 8.3 log CFU/mL or 7 log CFU/mouse.

For strain S14, the mice received (30 minutes pre-infection) a “preventive” antibiotic treatment (5 mg/kg or 30 mg/kg cloxacillin, or sterile saline) intraperitoneally. These doses were determined following an in-house PK/PD study.

For strain S39, the mice received (30 minutes pre-infection) a “preventive” antibiotic treatment (7 mg/kg or 45 mg/kg cloxacillin, or sterile saline) intraperitoneally. These doses were determined following an in-house PK/PD study. Serum cloxacillin concentrations under different conditions were measured by HPLC. All the results were integrated into a pharmacokinetic simulation software and then a PKPD software in order to estimate the different parameters related to the planktonic MIC (Cmax/MIC, AUC/MIC and T>MIC) and the MICb (Cmax/MICb, AUC/MICb and T>MICb).

At the same time, the control animals were given saline.

A commercial solution of cloxacillin (Orbenine® 1 g, France, Pfizer, France) was used. Then fixed anesthesia by intraperitoneal injection of a mixture of ketamine (50 mg/kg) and xylazine (10 mg/kg) was administered. The mixture of anesthetics was mixed as follows: 4 mL ketamine+2 mL xylazine+10 mL saline. Approximately 120 μL was injected IP in mice. The flanks were shaved and then disinfected (Betadine cycle). A 0.2 cm skin and subcutaneous incision was made sterile and a 1 cm segment of polyurethane catheter cut in two longitudinal parts (product code ES-04730, Arrow International) was inserted subcutaneously (2 cm away from the incision point). The bacterial inoculum was deposited at the same time. The volume deposited was 50 μL per mouse and the concentration tested was 10⁷ log CFU per catheter. The incision was then sutured and disinfected. The mice were then treated every two hours after the first preventive treatment for 10 hours with either 3 mg/kg or 15 mg/kg cloxacillin (for strain S14), 4 mg/kg or 25 mg/kg cloxacillin (for strain S39), or saline. These doses were pre-determined to simulate blood levels between the planktonic MIC and biofilm MICs of strains S14 and S39, respectively. The mice were euthanized at different post-infection times (12 h, 24 h and 30 h) by anesthesia followed by cervical dislocation. Catheter segments were removed. They were used for bacterial quantification after sonication.

Each polyurethane segment was individually and sterilely washed in an Eppendorf tube (3 successive washes with 300 μL sterile saline). After the last wash, the catheter was resuspended in 1 mL of physiological sterile water, placed in an ultrasound bath at room temperature for 3 min (AdvantageLab) and then vigorously vortexed to remove bacteria adhering to the catheter. Successive dilution cultures (pure, 10⁻², 10⁻³) of this bacterial suspension were then carried out on Chapman agars. The cultures were placed in an oven at 37° C. for 48 h before reading.

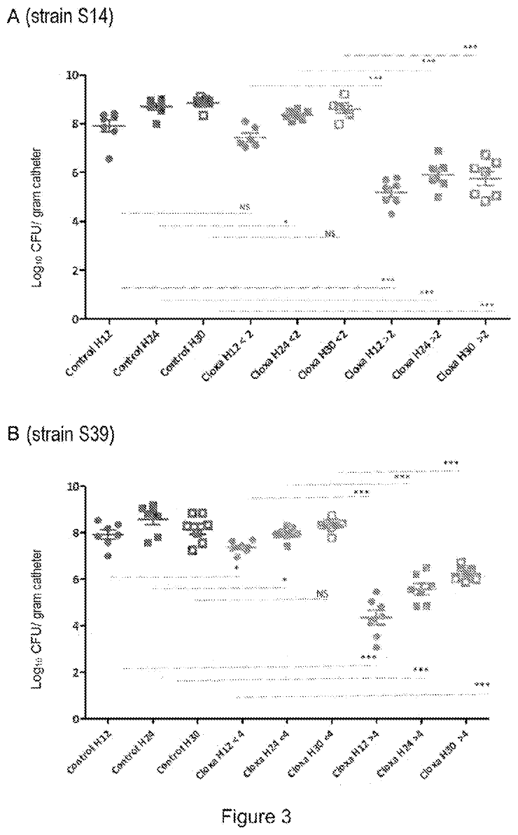

The results are shown in FIG. 3. FIG. 3 A, represents the enumeration of S14 strain bacteria attached to the implanted catheter based on the absence of cloxacillin (control), a concentration equal to the MIC or a concentration equal to the MICb. As shown in FIG. 3A, the concentration of cloxacillin under control and MIC conditions, shows the ineffectiveness of cloxacillin at the MIC dose to inhibit bacterial adhesion, at the 12 h, 24 h and 30 h measurement times. A one-way analysis of variance (ANOVA) supplemented by a post-hoc Bonferroni test was used. A difference was considered statistically significant if p<0.05 (*), p<0.01 (**) or p<0.001 (***). Conversely, for the 12 h, 24 h and 30 h times, cloxacillin treatment at the MICb dose showed a statistically significant difference from control (***). The results are expressed in log(CFU)/g catheter, colony-forming units (CFU), i.e. the number of bacterial colonies on a dish.

FIG. 3B shows the enumeration of S39 strain bacteria attached to the implanted catheter as a function of the absence of cloxacillin (control), a concentration equal to the MIC or a concentration equal to the MICb. As shown in FIG. 3B, the concentration of cloxacillin under control and MIC conditions, shows the ineffectiveness of cloxacillin at the MIC dose to inhibit bacterial adhesion, at the 12 h, 24 h and 30 h measurement times. A one-way analysis of variance (ANOVA) supplemented by a post-hoc Bonferroni test was used. A difference was considered statistically significant if p<0.05 (*), p<0.01 (**) or p<0.001 (***). Conversely, for the 12 h, 24 h and 30 h times, cloxacillin treatment at the MICb dose showed a statistically significant difference from control (***). The results are expressed in log(CFU)/g catheter, colony-forming units (CFU), i.e. the number of bacterial colonies on a dish.

This example clearly demonstrates that cloxacillin allows to inhibit the adhesion of bacteria to a surface, such as a prosthesis. Furthermore, this example clearly demonstrates that the usual concentrations of cloxacillin used as an antibiotic do not prevent/inhibit the adhesion of bacteria.

### Example 4

### In Vivo Inhibition of Biofilm Formation/Adhesion of Bacteria to a Surface in a Cloxacillin-Infected Skin Wound Healing Model

The objective of these trials was to test the efficacy of cloxacillin, at selected doses, in an in vivo model of mouse skin wound infection with strains of Staphylococcus aureus. The in vivo efficacy of specific doses of cloxacillin, derived from the in vitro MIC and MICb doses, was determined by measuring bacterial load (established as a biofilm) at 12 h, Day 5 and Day 11 post-infection, and by measuring wound healing at Day 5 and Day 11 post-infection (as a wound healing score).

In this example, the mice used were 7-week-old female C57BL/6 mice weighing between 20 and 22 g from Janvier Labs, Le Genest St Isle, 53941 St Berthevin, France. The number of mice used was 5 per condition tested.

In this example, the cloxacillin used was from a commercial cloxacillin solution (Orbenine® 1 g, France, Pfizer, France).

A Staphylococcus aureus strain, S14, was used. It has a minimum inhibitory concentration (MIC) for cloxacillin of 0.125 μg/mL and a minimum inhibitory concentration for biofilm formation (MICb) of 2 μg/mL.

The skin wound model was made as follows.

The concentration of S. aureus was adjusted in PBS to give a concentration of 10⁶ CFU in 10 μL (10⁸ CFU/mL). The mice, shaved on their backs, were anaesthetized with isoflurane gas and then 4 incisions were made on each side of the spine, defining 2 areas. In each mouse, only one of the areas was infected by subcutaneous injection of 10 μL of S. aureus solution, with the contralateral area serving as the uninfected control.

To test the effect of cloxacillin, 3 groups of mice received, 30 minutes pre-infection, a “preventive” treatment of 5 mg/kg cloxacillin (for mice subsequently receiving cloxacillin at 3 mg/kg) or 30 mg/kg cloxacillin (for mice subsequently receiving cloxacillin at 15 mg/kg) or sterile saline intraperitoneally. These doses were determined in an in-house PK/PD study.

Serum cloxacillin concentrations for different conditions were measured by HPLC according to a conventionally described protocol (see, for example, Jonsson et al., “Cloxacillin concentrations in serum, subcutaneous fat, and muscle in patients with chronic critical limb ischemia” Eur J Clin Pharmacol, (2014) 70(8):957-63). All the results were integrated into a pharmacokinetic simulation software and then a PKPD software in order to estimate the different parameters related to the planktonic MIC (Cmax/MIC, AUC/MIC and T>MIC) and the MICb (Cmax/MICb, AUC/MICb and T>MICb).

At the same time, the control animals, 5 mice, were given saline.

The mice were then treated every two hours after the first preventive treatment for 10 hours with cloxacillin at 3 mg/kg or 15 mg/kg or saline. These doses were pre-determined to simulate blood levels between the planktonic MIC and the biofilm MIC of strain S14.

Three groups of mice were used, a first group being sacrificed at t+12 h after infection to measure the bacterial load. A second group was treated identically and sacrificed 5 days after infection and a third group, treated identically, was sacrificed 11 days after infection. Each day, a healing score is established on the animals, and on the sacrificed animals, a count of the bacteria installed is made (at D5 and D11). The score is established as follows, assigning a value between 0 and 3.

0: complete healing;

1: wound not completely healed, with the presence of a scab;

2: red, unhealed wound (inflammation);

3: yellow, unhealed wound (presence of pus).

The skin at the wounds is cut, rinsed in PBS to remove non-adhered bacteria and then the tissue is homogenized in Precellys tubes using glass beads. The resulting homogenate was diluted serially, and different dilutions were spread on TSA media plates and cultured overnight at 37° C. Colonies (CFU) were quantified for each dilution and the bacterial load at the wound site was determined.

Results

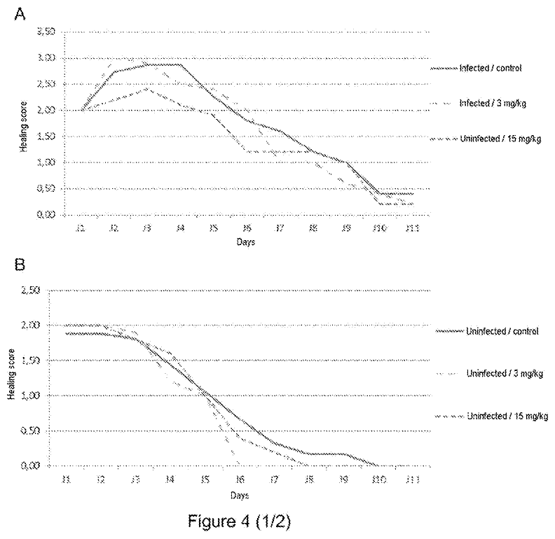

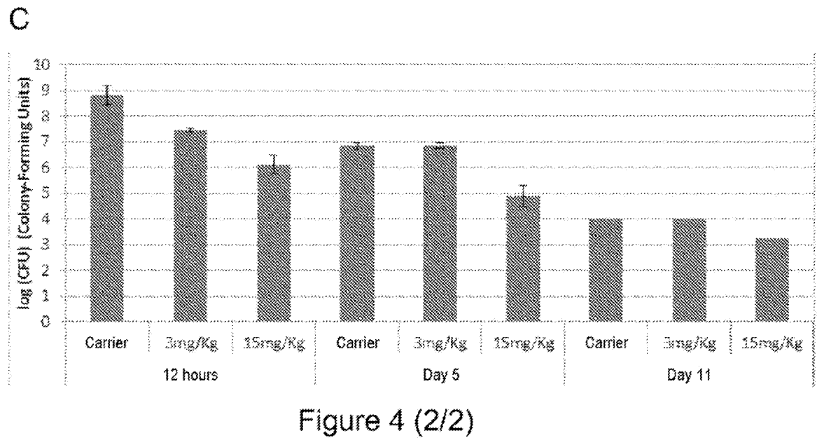

FIG. 4A shows the measurement of the healing score in infected mice treated with either cloxacillin corresponding to the MIC of strain S14 (3 mg/kg), cloxacillin corresponding to the MICb dose (15 mg/kg), or saline (vehicle).

FIG. 4B shows the measurement of the healing score in uninfected (control) mice treated with either cloxacillin corresponding to the MIC of strain S14 (Infected/3 mg/kg), cloxacillin corresponding to the MICb dose (Infected/15 mg/kg), or saline (Infected/Control).

FIG. 4C shows the count of bacteria in the wound. Their number is conventionally expressed in colony-forming units (CFU) corresponding to the number of colonies counted on a Petri dish.

It is evident from FIG. 4A that, at least up to day 6, the wound-healing score in infected animals decreases more rapidly in animals receiving the dose of cloxacillin intended to inhibit biofilm formation, thus indicating more effective wound-healing. The curve corresponding to the cloxacillin condition at the MIC dose overlaps with that corresponding to the cloxacillin-free control, showing the absence of cloxacillin effect on wound healing at this dose.

FIG. 4B shows that cloxacillin does not have a per se effect on wound healing in the absence of infection. At the same time, counting the bacteria on the wound after 12 h, 5 days and 11 days (FIG. 4C), shows that cloxacillin administration at the MICb dose of the strain (15 mg/kg) significantly decreases the bacterial load at the wound site (logio6.1 vs. logio8.82 for the control at 12 h), a decrease of almost 3logio, then the bacterial load continues to decrease faster than the control conditions without cloxacillin and cloxacillin at the MIC dose while the MICb dose of cloxacillin leads, on day 5, to a measurement of logio4.9 for the MICb dose versus logio6.86 for the MIC condition and logio6.84 for the control. The change parallels, from day 5 onwards, the control conditions without cloxacillin and cloxacillin at the MIC dose suggests that it is the immune system that is responsible for the decrease in bacterial load.

This example shows the inhibitory effect of Staphylococcus aureus installation at the wound site (biofilm formation) by cloxacillin at the MICb dose, which correlates with better wound site healing, and leads to the conclusion that the healing promoting effect of cloxacillin at the MICb dose of the strain is due to the inhibition of the installation of the strain. The administration of cloxacillin at a dose that is effective as an antibiotic (MIC dose), on the other hand, has no effect.

### LIST OF REFERENCES

1. Costerton et al., “Bacterial Biofilms: a common cause of persistent infections”. Science 1999; 284, 1318-1322
2. Hoiby, N. et al., “Antibiotic resistance of bacterial biofilms.” International Journal of

Antimicrobial Agents, 2010; (35): 322-332
3. Morgenstern et al., J Orthop Res. 2016 November; 34(11):1905-1913) “Biofilm formation increases treatment failure in Staphylococcus epidermidis device-related osteomyelitis of the lower extremity in human patients.”
4. Tunney et al., J Orthop Res. 2007 January; 25(1):2-10 “Biofilm Formation by Bacteria Isolated from Retrieved Failed Prosthetic Hip Implants in an In vitro Model of Hip Arthroplasty Antibiotic Prophylaxis
5. Trouillet-Assant et al., Cell Microbiol. 2016 October; 18(10):1405-14 “Adaptive processes of Staphylococcus aureus isolates during the progression from acute to chronic bone and joint infections in patients”
6. Valour et al., BMC Infect Dis. 2014 Aug. 16; 14:44 “Determinants of methicillin-susceptible Staphylococcus aureus native bone and joint infection treatment failure: a retrospective cohort study”
7. Singh and Mishra, 2012 Asian Journal of Plant Science and Research, 2 (3):350-354, “Synergistic and antagonistic actions of antibiotics against biofilm forming Staphylococcus aureus”,
8. Hoffman et al., 2005, Nature, 436: 1171-1175 “Aminoglycoside antibiotics induce bacterial biofilm formation”.
9. Vuotto et al., 2016, Pathod Dis, 74, doi: 10.1093/femspd/ftv114), “Sub-inhibitory concentrations of metronidazole increase biofilm formation in Clostridium difficile strains”
10. Mlynek et al., 2016, Antimicrob Agents Chemother., 60:2639-51 “Effects of Low-Dose Amoxicillin on Staphylococcus aureus USA300 Biofilms”
11. Mirani et al., 2011, J Basic Microbiol., 51:191-5 “Effect of sub-lethal doses of vancomycin and oxacillin on biofilm formation by vancomycin intermediate resistant Staphylococcus aureus”
12. Kaplan, 2011, Int J Artif Organs 34: 737-751). “Antibiotic-induced biofilm formation”

1. A method for inhibiting the formation and/or development of a biofilm on a surface, the method comprising:
applying cloxacillin at a concentration from 25 mg/kg on the surface.

2. The method of claim 1, wherein the concentration is from 25 mg/kg to 100 mg/kg.

3. The method of claim 1, wherein the surface is an abiotic surface.

4. The method of claim 1, wherein the concentration is from 25 mg/kg to 50 mg/kg.

5. A surface treated by the method of claim 1.

6. A method for preventing bacterial tolerance and/or resistance and/or multi-resistance related to the formation and/or development of a biofilm on a surface, the method comprising:
applying cloxacillin at a concentration from 25 mg/kg on the surface.

7. The method of claim 6, wherein the surface is a biotic surface.

8. A pharmaceutical composition for the prevention of bacterial biofilm formation on a surface comprising:
0.4% to 1.6% in weight percentage cloxacillin, based on the total weight of the pharmaceutical composition; and
a pharmaceutically acceptable carrier, an adjuvant or a vehicle.

9. The method of claim 13, wherein the concentration is from 25 to 50 mg/kg.

10. The method of claim 13, wherein the surface is a biotic surface.

11. A method for the prevention of bacterial biofilm formation in wounds and/or infections, the method comprising:
applying cloxacillin at a concentration from 25 mg/kg on the wounds and/or infections.

12. A method for the prevention of bacterial biofilm formation in wounds and/or infections, the method comprising:
applying the pharmaceutical composition of claim 8 on the wounds and/or infections,
wherein the cloxacillin is at a concentration from 25 mg/kg.

13. A method for the prevention of bacterial biofilm formation on a surface, the method comprising:
applying the pharmaceutical composition of claim 8 on the surface,
wherein the cloxacillin is at a concentration from 25 mg/kg.

14. The method of claim 13, wherein the surface is an abiotic surface.
